(19) United States
(12) Patent Application Publication
(10) Pub. No.: US 2023/0191114 A1
(43) Pub. Date: Jun. 22, 2023

(54) SYSTEMS AND METHODS FOR BACTERIAL BIOFILM INACTIVATION

(71) Applicant: OLD DOMINION UNIVERSITY RESEARCH FOUNDATION, Norfolk, VA (US)

(72) Inventors: Claudia MURATORI, Norfolk, VA (US); Erin B. PURCELL, Norfolk, VA (US); Asia POUDEL, Norfolk, VA (US); Adenrele M. OLUDIRAN, Norfolk, VA (US)

(21) Appl. No.: 17/996,104

(22) PCT Filed: Feb. 12, 2021
(86) PCT No.: PCT/US2021/017790

§ 371 Date: Oct. 13, 2022

Related U.S. Application Data
(60) Provisional application No. 63/011,178, filed on Apr. 16, 2020.

(51) Int. Cl. A61N 1/04; A61N 1/32; A61N 1/36

(52) U.S. Cl. CPC A61N 1/0476; A61N 1/328; A61N 1/36034; A61B 2018/00732

(57) ABSTRACT

Methods and apparatuses are described herein for inactivation of bacterial biofilms using sub-microsecond pulsed electric field application to an affected surface or region. In some examples, a bacterial biofilm may be inactivated while planktonic bacteria in the vicinity of the biofilm are not inactivated. These methods and systems provide an electrical-based therapeutic modality for which bacteria in biofilms may have difficulty developing resistance, unlike antibiotic therapies.

### CROSS REFERENCE TO RELATED APPLICATIONS

This patent application claims priority to U.S. provisional patent application No. 63/011,178, filed on Apr. 16, 2020, titled “SYSTEMS AND METHODS FOR BACTERIAL BIOFILM INACTIVATION,” herein incorporated by reference in its entirety.

### STATEMENT AS TO FEDERALLY SPONSORED RESEARCH

This invention was made with Government support under Grant No. K22 AI118929-01 awarded by NIH/NIAID. The Government has certain rights in the invention.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present disclosure relates to treatment of bacterial biofilms by the application of pulsed electric fields, such as sub-microsecond electrical pulses, e.g., a sub-microsecond pulsed electric field (e.g., nanosecond pulsed electric field).

### BACKGROUND

Bacterial biofilms are structured microbial communities formed on biotic or abiotic surfaces, and may be involved in a significant percentage of microbial infections. Development of bacteria biofilms on medical devices, such as vascular catheters, prosthetic joints, and cardiac pacemakers, may cause the most refractory modern bacterial diseases. Bacteria in biofilms are embedded within a self-produced extracellular matrix (ECM) consisting of nucleic acids, polysaccharides, and proteins and exhibit dramatically reduced susceptibility to antimicrobial agents as compared to planktonic cells of the same microorganism. New improved methods are needed to inactivate bacterial biofilms.

### SUMMARY OF THE DISCLOSURE

The present disclosure relates to improved methods and apparatuses (e.g., systems and methods) of inactivating biofilms by application of sub-microsecond pulsed electric field therapy. For example, described herein are methods of inactivating bacterial biofilms. Bacteria in biofilms are embedded within a self-produced extracellular matrix (ECM) consisting of nucleic acids, polysaccharides, and proteins, and can lead to refractory infections difficult to eradicate and susceptible to increasing antimicrobial resistance generally.

As used herein, inactivating a biofilm may include disrupting, destroying, killing, or reducing the biofilm (e.g., bacterial biofilm. In some examples disrupting the biofilm may include treating an infection. In some examples, disrupting the biofilm may include treating a condition (e.g., illness, malady, etc.) arising from a biofilm. For example, described herein are methods of inactivating a bacterial biofilm comprising applying a sub-microsecond pulsed electric field comprising an electric field strength from 0.1 kV/cm to 9.9 kV/cm to a surface comprising a bacterial biofilm until the bacterial biofilm is inactivated; in some examples, these methods may include limit inactivation of planktonic bacteria in proximity to the bacterial biofilm. In some examples, the methods described herein are methods of treating acne. These methods may be cosmetic methods for treating the skin, including non-therapeutic (e.g., non-medical) treatments.

The methods and devices of the present disclosure use sub-microsecond pulsed electric field as a potential alternative for microbial inactivation within bacterial biofilms. Since the technique employs electrical energy, it is difficult for bacteria to develop resistance. Resistance development is a major failing of antimicrobial biomolecule/molecule treatments of abiotic surfaces or antimicrobial drug therapies for bacterial biofilm infections on, within or in proximity to biotic surfaces.

According to the present disclosure, Applicants have applied the use of pulsed electric fields of nanosecond duration (pulse duration range of less than about 1 ms, e.g., less than about 900 ns, less than about 800 ns, less than about 700 ns, less than about 500 ns, from about 1 ns to about 999 ns, from about 1 ns to 500 ns, from about 10 ns to 500 ns, from about 50 ns to 500 ns, from about 100 ns to 500 ns, from about 1 ns to 400 ns, from about 1 ns to 300 ns, etc.), amplitude from about 0.1 to 100 kV/cm, and pulse number about 1 to about 1000 to provide effective cell killing effects within bacterial biofilms. Application of sub-microsecond (e.g., nanosecond, picosecond, etc.) pulsed electric field may be dosed at levels which provide effective cell killing within the biofilm while not killing planktonic bacteria similarly dosed. The heightened susceptibility of bacterial biofilms to sub-microsecond pulsed electric field relative to the planktonic bacteria commensals is demonstrated for the first time here. This therapeutic modality may provide a solution to difficult therapeutic problems when attempting to limit damage to the tissue of a subject surrounding the bacterial biofilm infection as well as maintaining commensal planktonic bacterial populations necessary for a healthy microbiome.

Accordingly, a method of inactivating a bacterial biofilm is provided including applying a sub-microsecond pulsed electric field having an electric field strength from 0.1 kV/cm to 100 kV/cm to a surface including a bacterial biofilm. In some examples, a method of inactivating a bacterial biofilm is provided including applying a sub-microsecond pulsed electric field having an electric field strength from 0.1 kV/cm to about 50 kV/cm to a surface (e.g., in vivo) including a bacterial biofilm. In some examples, a method of inactivating a bacterial biofilm is provided including applying a sub-microsecond pulsed electric field having an electric field strength from 0.1 kV/cm to about 9.9 kV/cm to a surface including a bacterial biofilm. The surface may be internal or external. The sub-microsecond pulsed electric field may include from 5 pulses to 1000 pulses.

In some examples, e.g., in-vivo, the sub-microsecond pulsed electric field may include an electric field strength from about 0.1 kV/cm to about 40.0 kV/cm, or from about 0.5 kV/cm to about 30 kV/cm. In some other examples, the pulsed electric field may include an electric field strength from about 0.1 kV/cm to about 5.0 kV/cm. In yet other examples, the pulsed electric field may include an electric field strength from about 0.5 kV/cm to about 3.5 kV/cm.

In some examples, the bacterial biofilm may include Gram-negative, Gram-positive, or mixed-species bacterial biofilms. In some examples, the bacterial biofilm may include Cutibacterium acnes (formerly Propionibacterium acnes).

In some examples, applying the pulsed electric field may include applying the pulsed electric field in proximity to a surface including the bacterial biofilm.

In some examples, the surface including the bacterial biofilm may be biotic. In some examples, the surface including the bacterial biofilm may include an epithelial surface. In some examples, the surface including the bacterial biofilm may include an epithelial surface and epithelial tissues underlying the epithelial surface. In some examples, applying the sub-microsecond pulsed electric field may include applying an applicator designed to deliver the sub-microsecond pulsed electric field in proximity to the surface.

In some examples, the sub-microsecond pulsed electric field may have a pulse duration from 1 nanosecond to 999 nanoseconds. In some other examples, the sub-microsecond pulsed electric field may have a pulse duration from 10 nanoseconds to 500 nanoseconds. In some other examples, the sub-microsecond pulsed electric field may have a pulse duration from 50 nanoseconds to 400 nanoseconds or 100 nanoseconds to 400 nanoseconds.

In some examples, the sub-microsecond pulsed electric field may have a frequency from 0.5 Hz to 3 MHz. In some examples, the sub-microsecond pulsed electric field may have a frequency of 1 Hz, 5 Hz, 100 Hz, 1 kHz, or 10 kHz. In some other examples, the sub-microsecond pulsed electric field may have a frequency from 1 Hz to 10 Hz.

In some examples, a population of bacteria including the bacterial biofilm may be reduced by a factor of 10² to 10⁴. In some examples, a population of bacteria including the bacterial biofilm may be reduced by a factor of 10² to 10⁴ and a population of the planktonic bacteria may be reduced by less than a factor of 10.

In some examples, the method may further include contacting the surface comprising the bacterial biofilm with lysozyme prior to applying the sub-microsecond pulsed electric field. In some examples, contacting the surface including the bacterial biofilm may include contacting the surface with lysozyme at a concentration from at least 1 mg/ml to 10 mg/ml within a liquid medium. In some examples, the surface including the bacterial biofilm may be contacted with lysozyme for a period of time from 10 min to 1 h.

In another aspect, a method for inactivating a bacterial biofilm is provided, including applying a pulsed electric field designed to inactivate bacteria including the bacterial biofilm while limiting inactivation of planktonic bacteria in proximity to the bacterial biofilm. In some examples, the planktonic bacteria may include commensal bacteria.

In some examples, the pulsed electric field may include an electric field strength of at least 0.1 kV/cm. In some examples, the pulsed electric field may include an electric field strength from 0.1 kV/cm to 9.9 kV/cm. In some other examples, the pulsed electric field may include an electric field strength from 0.5 kV/cm to 3.5 kV/cm.

In some examples, the pulsed electric field may include a pulse duration from 10 nanoseconds to 900 nanoseconds. In some other examples, the pulsed electric field may include a pulse duration from 10 nanoseconds to 500 nanoseconds. In yet other examples, the pulsed electric field may include a pulse duration from 100 nanoseconds to 400 nanoseconds.

In some examples, the pulsed electric field may include from 5 pulses to 1000 pulses. In some examples, the pulsed electric field may have a frequency from 1 Hz to 3 MHz. In some examples, the pulsed electric field may have a frequency from 0.5 Hz to 10 Hz or from 1 Hz to 10 Hz.

In some examples, the bacterial biofilm may include a Gram-positive bacterial biofilm, a Gram-negative bacterial biofilm or a bacterial biofilm including both Gram-positive and Gram-negative bacteria. In some examples, the bacterial biofilm may include C. acnes bacteria.

In some examples, applying the pulsed electric field may include applying the pulsed electric field in proximity to a surface including the bacterial biofilm.

In some examples, the surface including the bacterial biofilm is biotic. In some examples, the surface including the bacterial biofilm may include an epithelial surface. In some examples, the surface including the bacterial biofilm may include an epithelial surface and epithelial tissues underlying the epithelial surface.

In some examples, applying the pulsed electric field may include applying an applicator designed to deliver the pulsed electric field in proximity to the surface.

In some examples, a population of bacteria including the bacterial biofilm may be reduced by a factor of 10² to 10⁴. In some examples, a population of bacteria including the bacterial biofilm may be reduced by a factor of 10² to 10⁴ and a population of the planktonic bacteria may be reduced by less than a factor of 10.

In some examples, the method may further include contacting the bacterial biofilm with lysozyme prior to applying the sub-microsecond pulsed electric field. In some examples, contacting the bacterial biofilm may include contacting the bacterial biofilm with lysozyme at a concentration from at least 1 mg/ml to 10 mg/ml within a liquid medium. In some examples, the bacterial biofilm may be contacted with lysozyme for a period of time from 10 min to 1 h.

In another aspect, a method of treating acne in a region of skin of a subject is provided, including applying a sub-microsecond pulsed electric field to the region of the skin, where the sub-microsecond pulsed electric field is designed to inactivate bacteria including a C. acnes bacterial biofilm while limiting cellular damage to surrounding tissues in the region of the skin. In some examples, the method may include limiting inactivation of commensal planktonic bacteria in the region.

In some examples, the sub-microsecond pulsed electric field may include an electric field strength from 0.1 kV/cm to 100 kV/cm or from 0.1 kV/cm to 50 kV/cm. In some other examples, the sub-microsecond pulsed electric field may include an electric field strength from 0.1 kV/cm to 9.9 kV/cm. In some other examples, the sub-microsecond pulsed electric field may include an electric field strength from 0.1 kV/cm to 5.0 kV/cm. In yet other examples, the sub-microsecond pulsed electric field may include an electric field strength from 0.5 kV/cm to 3.5 kV/cm.

In some examples, the sub-microsecond pulsed electric field may include a pulse duration of less than 1000 nanoseconds. In some other examples, the sub-microsecond pulsed electric field may include a pulse duration from 10 nanoseconds to 500 nanoseconds. In yet other examples, the sub-microsecond pulsed electric field may include a pulse duration from 100 nanoseconds to 400 nanoseconds.

In some examples, the sub-microsecond pulsed electric field may include from 5 pulses to 1000 pulses. In some examples, the sub-microsecond pulsed electric field may have a frequency from 1 Hz to 3 MHz. In some examples, the sub-microsecond pulsed electric field may have a frequency from 0.5 Hz to 10 Hz or from 1 Hz to 10 Hz. In some other examples, the sub-microsecond pulsed electric field may have a frequency from 1 Hz to 5 Hz.

In some examples, the method may further include contacting the region of the skin with lysozyme prior to applying the sub-microsecond pulsed electric field. In some examples, contacting the region of the skin may include contacting the region of the skin with lysozyme at a concentration from at least 1 mg/ml to 10 mg/ml within a liquid medium. In some examples, the region of the skin may be contacted with lysozyme for a period of time from 10 min to 1 h.

Also described herein are apparatuses, e.g., systems, for performing any of the methods described herein. For example, described herein are systems for inactivating a bacterial biofilm that include: an applicator comprising a set of electrodes configured to be placed in proximity to the bacterial biofilm; a pulse generator configured to generate a sub-microsecond pulse; and a controller comprising one or more processors, wherein the controller comprises a machine-readable medium storing instructions that, when executed by the one or more processors, cause the pulse generator to apply a train of sub-microsecond electrical pulses having an electric field strength of between 0.1 kV/cm to 100 kV/cm to the applicator when a biofilm is between the set of electrodes.

In some examples the applicator is a skin-contacting applicator. For example, an applicator for delivering energy to disrupt a biofilm on the surface of a tissue (e.g., skin tisuse0 may have a first skin-contacting surface adjacent to a first electrode, a second skin-contacting surface adjacent to a second electrode, and a therapy region between the first and second electrodes and configured to apply an electric field between the first and second electrodes. The skin-contacting surface may be conformable. For example, the skin-contacting surface may be configured to be held against the skin to hold the region between the first and second electrodes taut. The first and second electrodes may contact the skin or may be configured to not contact the skin but may be held adjacent to the skin. In some examples the skin-contacting surfaces may be part of an arm, prong or extension of the applicator. In some example, the skin-contacting surfaces are on either side of an opening or window of the applicator. In general, the methods and apparatuses, including applicators, described herein are not limited to tissue, but may be used for reducing (including in some examples, eliminating) a biofilm on non-biological surfaces.

The first and second electrodes may be positioned between the structures (arms, prongs, sides, etc.) forming the first and second contacting surfaces. In case of the use for the treatment of tissue, these could be tissue- or skin-contacting surfaces, e.g., on either side of a therapy region. The therapy region may be referred to as a therapy window. The therapy region may be open so that the tissue surface (e.g., skin surface) may be visible between the electrodes. The first and second electrodes may be configured to apply an electric field between them so as to apply energy to a biofilm on the surface of the tissue (e.g., skin), as described herein.

The skin-contacting surfaces may be more generally described as tissue-contacting surfaces.

For example, a system for inactivating a bacterial biofilm may include: an applicator including a set of electrodes designed to be placed in proximity to the bacterial biofilm; one or a plurality of pulse generators, where each pulse generator is designed to generate a sub-microsecond pulse; and a controller including one or more processors designed to provide a train of sub-microsecond pulses to the applicator, where the controller further may include a machine-readable tangible medium storing instructions for causing the one or more processors to execute operations for: applying a train of sub-microsecond electrical pulses including an electric field strength from 0.1 kV/cm to 100 kV/cm. In some examples, the machine-readable tangible medium may store instructions for causing the one or more processors to execute operation for applying a train of sub-microsecond electrical pulses comprising an electric field strength from 0.1 kV/cm to 9.9 kV/cm.

In some examples, the instructions may include instructions causing an electric field strength from 0.1 kV/cm to 5.0 kV/cm. In some other examples, the instructions may include instructions causing an electric field strength from 0.5 kV/cm to 3.5 kV/cm. In some other examples, the instructions may include instructions causing an electric field strength from 0.5 kV/cm to 75 kV/cm, 0.5 kV/cm to 50 kV/cm, or 0.5 kV/cm to 30 kV/cm.

In some examples, the instructions may include instructions causing a pulse duration from 10 nanoseconds to 900 nanoseconds. In some other examples, the instructions may include instructions causing a pulse duration from 10 nanoseconds to 500 nanoseconds. In yet other examples, the instructions may include instructions causing a pulse duration from 100 nanoseconds to 400 nanoseconds.

In some examples, the instructions may include instructions causing a train of sub-microsecond electrical pulses from 10 pulses to 1000 pulses. In some examples, the instructions may include instructions causing a frequency from about 0.5 Hz to about 3 MHz. In some other examples, the instructions may include instructions causing a frequency from about 0.5 Hz to about 10 Hz or about 1 Hz to about 10 Hz.

Any of the apparatuses (e.g., systems) described herein may be configured as systems for treating acne.

### BRIEF DESCRIPTION OF THE DRAWINGS

The novel features of the present disclosure are set forth with particularity in the claims that follow. A better understanding of the features and advantages of the present disclosure will be obtained by reference to the following detailed description that sets forth illustrative examples, in which the principles of the disclosure are utilized, and the accompanying drawings of which:

FIGS. 1A and 1B are examples of graphical representations of viability of C. acnes cells after sub-microsecond pulsed electric field treatment according to an example of the disclosure.

FIG. 2 is an example of a graphical representation of the extent of cell killing by sub-microsecond pulsed electric field of C. acnes cells in suspension and within a biofilm according to an example of the disclosure.

FIGS. 3A and 3B are examples of graphical representations of experimental details of cell killing of undisturbed biofilms containing C. acnes cells by sub-microsecond pulsed electric field according to an example of the disclosure.

FIG. 3C is another example of graphical representation of the extent of cell killing of undisturbed biofilms containing C. acnes cells by sub-microsecond pulsed electric field according to an example of the disclosure.

FIG. 4A is an example of a graphical representation of the extent of cell killing of planktonic C. acnes cells pre-treated with lysozyme.

FIG. 4B is an example of a graphical representation of the extent of cell killing of biofilm-contained C. acnes cells pre-treated with lysozyme.

FIGS. 5A and 5B show one example of a pulsed electric field apparatus including a pulse generator, an applicator and a controller controlling the pulse generator.

FIG. 5C illustrates one example of a trapezoidal pulse of 280 ns duration, 28 kV/cm produced by the example pulse generator of FIGS. 5A-5B; this pulse may be one of a train of pulses delivered to the applicator (shown in FIG. 5B as a cuvette).

FIGS. 5D and 5E show side and top views, respectively, of another example of an applicator configured as a skin applicator.

FIGS. 6A-6D illustrates C. acnes growth condition optimization for planktonic vs. biofilms. In FIG. 6A, C. acnes cells from a frozen glycerol stock were aliquoted into TY medium, let it grow overnight, and plated on the indicated agar plates. Cells on BHIS, TY, and chocolate agar plates were plated at a 10⁵ dilution while cells on blood agar plates were seeded at a 10⁶ dilution (the scale bar is 5 mm). FIG. 6B is a graph showing the growth of planktonic C. acnes over 72 h. FIG. 6C. shows images of crystal violet staining of C. acnes biofilms grown for 72 h on glass coverslips with no coating (CTRL) and coated with 0.1% fibronectin (FIB), 0.1% poly-D-lysine (PDL), or 0.1% poly-L-lysine (PLL). FIG. 6D. illustrates an example of biofilm (BF) and liquid culture viability after 60 minutes incubation in an electroporation cuvette in anaerobic (circle) or aerobic (square) conditions. Mean+/−s.e. These C. acnes bacteria grown as shown in FIGS. 6A-6D provided a bacterial model for the methods and apparatus described herein.

FIG. 7 is a graph illustrating the synergistic cytotoxicity from combination of lysozyme treatment and nanosecond pulsed electrical field treatment as described herein. In FIG. 7, planktonic C. acnes cells in exponential phase were treated with either nanosecond pulsed electrical field treatment (e.g., 1000 or 2000, 280 ns, 28 kV/cm, 5 Hz) or lysozyme (LY; 1 or 10 mg/ml) for 1 h, or both nanosecond pulsed electrical field treatment and lysozyme, and viability was measured at 72 h post treatment. Mean+/−s.e., n=3-5.

### DETAILED DESCRIPTION

Bacteria in biofilms are embedded within a self-produced extracellular matrix (ECM) consisting of nucleic acids, polysaccharides, and proteins. Bacteria in biofilms exhibit dramatically reduced susceptibility to antimicrobial agents as compared to planktonic cells of the same microorganism, thus rendering bacterial biofilm infections particularly resistant to existing therapies. Several potential mechanisms implicated in biofilm resistance have been proposed, including: restricted penetration through the biofilm matrix, presence of antimicrobial inactivating enzymes, slow growth rate, stress response, and altered gene expression.

The present disclosure is directed to new methods and systems of using Sub-microsecond pulsed electric field (e.g., nanosecond pulsed electric field, picosecond pulsed electric field, etc.) for effective microbial inactivation within bacterial biofilms. Since the sub-microsecond pulsed electric field employs physical forces, e.g., electrical, it is difficult for resistance to develop, which is a major failing of antimicrobial treatments of abiotic surfaces or antimicrobial drug therapies for bacterial biofilm infections on, within or in proximity to biotic surfaces.

According to one aspect, it was discovered that pulsed electric fields of sub-microsecond duration, such as nanosecond duration (between 1 ns and about 1000 ns, e.g., between about 1 ns and about 900 ns, between about 1 ns and about 800 ns, between about 1 ns and about 600 ns, between about 1 ns and about 500 ns, between about 10 ns and about 500 ns, between about 50 ns and about 500 ns, between about 100 ns and about 500 ns, etc.) and amplitude from 0.1 to 100 kV/cm provide effective cell killing effects within bacterial biofilms. Further, Applicants have surprisingly discovered that application of sub-microsecond pulsed electric field may be dosed at levels which provide effective cell killing within the biofilm while not killing planktonic bacteria similarly dosed. The heightened susceptibility of bacterial biofilms to sub-microsecond pulsed electric field relative to the planktonic bacteria commensals has not been demonstrated before. This may provide a solution to difficult therapeutic problems when attempting to limit damage to the tissue of a subject surrounding the bacterial biofilm infection as well as maintaining commensal planktonic bacterial populations necessary for a healthy microbiome.

Without wishing to be bound by theory, the antibacterial activity of sub-microsecond pulsed electric field may result from direct damage to the cell plasma membrane and/or formation of reactive oxygen species within the biofilm environment, causing the bacteria within the biofilm to experience heightened concentration trapped by the biofilm. As described herein, experiments providing sub-microsecond pulsed electric field to bacteria within biofilms and planktonic bacteria of Cutibacterium (formerly Propionibacterium) acnes (C. acnes) was effective to kill about 90% of the bacteria within the biofilm, while not significantly affecting the planktonic phenotype bacteria. These results provided a first demonstration of greater sensitivity to an extracellular stress by bacteria in biofilms than when the bacteria are living in their equivalent planktonic form.

Accordingly, a method of inactivating a bacterial biofilm is provided, including applying a sub-microsecond pulsed electric field to a surface including a bacterial biofilm. In some examples, the surface may be an abiotic surface, such as a catheter, a stent, prosthetic joints, and the like. In some other examples, the surface may be a biotic surface such as the surface of an epithelial cell or tissue. In some examples, the biotic surface may be on or within the surface of skin of a subject. In some further examples, the biotic surface may be on, within, adjacent, or in a region in proximity to or underlying the biotic surface, such as skin or other epithelial surfaces. In some examples, the methods described herein treat epithelial tissue. The epithelial tissue need not be limited to tissue associated with or underlying skin but may be oral mucosal tissue, tissues lining the intestinal tract, tissue at a wound or surgical site, or adjoining a stent, prosthetic joint, and the like.

The method may include applying a pulsed electric field designed to inactivate bacteria including the bacterial biofilm while limiting inactivation of planktonic bacteria in proximity to the bacterial biofilm. The planktonic bacteria may commensal bacteria and may include other species of bacteria in addition to the bacteria within the biofilm causing the pathogenic infection.

The bacterial biofilm may include any kind of bacteria. In some examples, the bacterial biofilm includes a Gram-positive bacterial biofilm. While Gram-positive bacteria are known to be difficult to treat by conventional methods due to the nature of the peptidoglycan compositions of the cell wall, the methods and devices of the present disclosure using nanosecond pulsing may effectively treat Gram-positive bacteria biofilms. In some examples, the bacterial biofilm may include C. acnes bacteria. However, the method is not limited to treating Gram-positive bacterial biofilm infections, but may also be used to treat Gram-negative bacterial biofilm infections.

Applying the pulsed electric field to the surface may include applying the pulsed electric field in proximity to a surface including the bacterial biofilm, e.g., inserting electrode needles against, or in some examples, into tissues of the region near or in proximity to the surface. In some examples, applying the pulsed electric field may include applying an applicator designed to deliver the pulsed electric field in proximity to the surface, e.g., to tissues surrounding the surface, where bacterial biofilms may form.

In some examples, applying the pulsed electric field may cause a population of bacteria of the bacterial biofilm to be reduced by a factor of 10² to 10⁴. In some examples, while the bacteria of the bacterial biofilm are reduced a factor of 10² to 10⁴, a population of planktonic bacteria may be reduced by less than a factor of 10.

In some examples, the nanosecond, e.g. sub-microsecond, pulsed electric field may have an electric field strength of about 0.1 kV/cm to about 9.9 kV/cm; about 0.1 kV/cm to about 5.0 kV/cm; or about 0.5 kV/cm to about 3.5 kV/cm, or any value therebetween. In some examples, the sub-microsecond pulsed electric field may have a strength less than about 9.0 kV/cm; less than about 8.0 kV/cm; less than about 7.0 kV/cm; less than about 6.0 kV/cm; less than about 5.0 kV/cm; less than about 4.0 kV/cm; less than about 3.0 kV/cm; or less than about 2.0 kV/cm. In some other examples the sub-microsecond pulsed electric field may have a strength of about 0.1 kV/cm; about 0.4 kV/cm; about 0.6 kV/cm; about 0.8 kV/cm; about 1.0 kV/cm; about 1.5 kV/cm; about 2.0 kV/cm; about 3.0 kV/cm; about 4.0 kV/cm; about 5.0 kV/cm; about 6.0 kV/cm; or about 7.0 kV/cm.

In some other examples, such as for example, in-vivo applications, the nanosecond, e.g. sub-microsecond, pulsed electric field may have an electric field strength of about 0.1 kV/cm to about 100 kV/cm; about 0.1 kV/cm to about 75 kV/cm; about 0.1 kV/cm to about 50 kV/cm, about 0.1 kV/cm to about 40 kV/cm; or about 0.5 kV/cm to about 30 kV/cm, or any value therebetween. In some examples, the sub-microsecond pulsed electric field may have a strength less than about 100 kV/cm, less than about 50 kV/cm; less than about 45 kV/cm; less than about 40 kV/cm; less than about 30 kV/cm; less than about 25 kV/cm; less than about 20 kV/cm; or less than about 15 kV/cm. In some other examples the sub-microsecond pulsed electric field may have a strength of about 1.0 kV/cm; about 5.0 kV/cm; about 10 kV/cm; about 15 kV/cm; about 20 kV/cm; about 25 kV/cm; about 28 kV/cm; about 30 kV/cm; about 35 kV/cm; about 45 kV/cm; about 50 kV/cm, about 60 kV/cm; about 70 kV/cm; about 75 kV/cm; about 80 kV/cm; about 90 kV/cm; or about 100 kV/cm.

In some examples, the nanosecond, e.g. sub-microsecond, pulsed electric field may be applied in about 5 pulses to about 1000 pulses, or any number of pulses therebetween. In some examples, the train of pulses may include about 10, about 50, about 100, about 200, about 300, about 500, about 600, about 800, or about 900 or about 1000 pulses.

In some examples, the nanosecond, e.g. sub-microsecond, pulsed electric field may have a pulse duration from about 1 nanosecond to about 999 nanoseconds; about 10 nanoseconds to about 500 nanoseconds; about 50 nanoseconds to about 400 nanoseconds; about 100 nanoseconds to about 500 nanoseconds; about 200 nanoseconds to about 600 nanoseconds; or about 300 nanoseconds to about 700 nanoseconds. In some examples, the sub-microsecond pulsed electric field may have a pulse duration of about 10 nanoseconds; about 30 nanoseconds; about 50 nanoseconds; about 70 nanoseconds; about 100 nanoseconds; about 130 nanoseconds; about 150 nanoseconds; about 170 nanoseconds; about 190 nanoseconds; about 220 nanoseconds; about 250 nanoseconds; about 300 nanoseconds; about 400 nanoseconds; about 500 nanoseconds; about 600 nanoseconds; about 700 nanoseconds; about 800 nanoseconds; or about 900 nanoseconds; or any pulse duration therebetween.

The shape of the pulse may be any suitable shape, square, trapezoidal, etc., and have any suitable rise time (e.g., 10 ns, 15 ns, 20 ns) to deliver the pulsed electric field to the surface/tissue.

In some examples, the nanosecond, e.g. sub-microsecond, pulsed electric field may have a frequency from about 0.5 Hz to about 10 Hz; about 1 Hz to about 5 Hz; about 3 Hz to about 7 Hz; above 10 Hz, about 50 Hz, about 100 Hz, about 500 Hz, about 1 kHz, about 10 kHz, about 1 MHz, or for example, up to about 3 MHz, or any frequency therebetween.

One of skill in the field may successfully choose to vary the above elements to arrive at a successful treatment application.

### Therapeutic Application

Since the methods of the present disclosure of inactivating bacterial cells within a biofilm may avoid damaging nearby cells and/or may avoid damaging/killing nearby planktonic bacteria that may be beneficial, it may be a very powerful tool to address stubbornly treatment-resistant biofilm infections. Nearby planktonic bacteria may be commensal bacteria, and may include a number of other species, thus preserving commensal planktonic bacteria may be essential to the microbiome in maintaining homeostasis. While the treatment of acne is discussed at length here, other non-limiting examples of suitable therapeutic uses may be to treat surgical areas such as knee or joint surgeries where the incisions have become infected and the surgical sites need to be cleared of bacterial biofilm infection. Typically, the area is physically abraded, treated with antimicrobials and packed. This treatment is painful, does not always successfully inactivate the bacterial infection, and it is difficult for patients to repack the surgical site on their own. Moreover, the use of broad spectrum antibiotics can disrupt commensal gut microbiota and predispose patients to antibiotic-associated diarrhea (AAD) and Clostridioides difficile infection (CDI). Antibiotic stewardship to prevent the spread of antimicrobial resistance is a high public health priority, creating a need for non-antibiotic methods to treat bacterial infections. According to the present disclosure, sub-microsecond pulsed electric field applied to a cleaned but not abraded site may be more tolerable and more successful.

In another non-limiting application, during chemotherapy sequences for cancer treatment, patients may acquire painful and therapy shortening oral mucositis infections. Treatment of these oral infections may permit more rapid recovery, since systemic antibiotics are not needed, and the commensal oral bacteria may be spared.

In yet another non-limiting application, surgical interventions within the gastrointestinal tract, for example, for severe obstruction of the intestine, may leave suture sites which can become infected. ns-PEF application via endoscopic procedures may preferably clear bacterial biofilm infections at a suture site, while limiting damage to the flora in the G. I. tract. Such localized, physical disruption of site-specific infection could limit the use of broad-spectrum antibiotics, avoid antibiotic-induced disbiosis in the commensal gut microbiota, and contribute to antibiotic stewardship efforts.

One specific example of a therapeutic area where sub-microsecond pulsed electric field may be usefully employed is acne (acne vulgaris). Cutibacterium acnes formerly known as Propionibacterium acnes is a Gram-positive, non-spore former, facultative anaerobic rod-shaped biofilm forming bacterium that colonizes in human skin. It is an opportunistic pathogen associated with invasive skin infections such as acne vulgaris as well as medical device related infections (e.g., by adhering to surfaces of such medical devices).

Acne is a very common skin disorder amongst adolescents, where approximately 70% of adolescents have an outbreak of acne at some point in time. However, individuals of other age groups may also be affected and the prevalence of adult acne vulgaris is increasing, particularly for women of 25 yrs. of age and older. In the United States alone, the estimated direct health care cost for all kinds of skin related disease approaches 75 billion dollars, for which the treatment of acne vulgaris is a leading contributor of cost.

The most common treatments include the topical application of retinoids as well as systemic treatments, photosensitizers and natural approaches. However, the efficacy of these treatments is minimal and results in undesirable side effects such as hyperpigmentation, exfoliation and crusting. Further, in some rare cases the use of products containing benzoyl peroxide or salicylic acid can cause serious and potentially life threatening allergic reactions. In addition to conventional therapeutics, new phototherapy and thermal treatment strategies have been developed. Phototherapies may employ blue visible wavelength, red visible wavelength or a combined wavelength therapeutic exposure, and may include photosensitizers such as aminolevulinic acid or methyl aminolevulinate. However, high energy light exposure or thermal treatment may increase local temperatures enough to damage to tissue underlying the skin.

Antimicrobial compounds have been used as therapeutics but most strains of bacteria can develop resistance, and development of new antimicrobial compounds requires extensive and lengthy development- only to face similar problems of resistance development.

As mentioned above, biofilm forming cells are more resilient towards antimicrobial stress than planktonic cells. Antibiotic therapy against C acnes can become quite challenging, frequently leading to emergence of antibiotic resistance. Biofilm formation may be one important factor contributing to the chronic pathophysiology of acne vulgaris. The disposition of bacterial biofilms of C acnes on, in or within the surface of skin (e.g., epithelial surfaces) and underlying regions in proximity thereto make treatment using sub-microsecond pulsed electric field, as described here and in the Examples, a desirable approach which avoids the issues of drug resistance. Such localized intervention would minimize off-target killing of commensal skin microbes, which may prevent acne. Nearby populations of beneficial skin microbiota would not be disrupted by sub-microsecond pulsed electric field treatment as they would be by application of topical antibiotics.

As discussed in detail herein, Applicant has demonstrated that use of sub-microsecond pulsed electric field may permit selective inactivation of the C. acnes biofilm contributing to chronic acne infections without damaging the co-habiting commensal bacteria in the skin as well as limiting damage to the epithelial tissues of the skin.

Additionally, the use of lysozyme was explored to increase the susceptibility of Gram-positive bacteria, such as C. acnes, to electrotransformation by sub-microsecond pulsed electric field due to the structure and density of their cell walls. Increasing the fragility of the cell wall may increase the transformation efficiency significantly. Among these cell-wall-weakening agents, lysozyme is a naturally occurring enzyme found in bodily secretions such as tears, saliva, and milk. It is considered a part of the innate immune system in most mammals and degrades peptidoglycan in the bacterial cell wall. Lysozyme is an attractive adjunct for sub-microsecond pulsed electric field, as it is currently approved for use in skin care and as a safe adjunct to antifungals. Previous reports indicated that pretreatment with Lysozyme-triclosan complexes enhanced bactericidal activity against several strains of Gram-positive and Gram-negative bacteria. As discussed in detail in Experiment 4, incubation of planktonic C. acnes with lysozyme at several concentrations reduced C. acnes viability upon subsequent treatment with sub-microsecond pulsed electric field. Thus, in some examples, a potential combination therapy which can address both planktonic populations of C. acnes and biofilm-contained C. acnes bacteria may include both lysozyme pretreatment, followed by subsequent sub-microsecond pulsed electric field. The lysozyme-triclosan complex may be present at a concentration from at least 1 mg/ml, about 2 mg/ml, about 5 mg/ml, about 7 mg/ml, about 10 mg/ml, or about 15 mg/ml. The lysozyme-triclosan complex may contact the surface including a bacterial biofilm, e.g., such as skin or an abiotic surface, for about 10 min, about 20 min, about 30, about 40 min, about 1 h, about 2 h, or more.

Treatment may be made using a sub-microsecond pulsed electric field applicator that may be handheld or, in some examples, the sub-microsecond pulsed electric field applicator may be coupled to a movable arm of an automated system, such as a robotic system. For example, the applicator and/or a controller controlling the pulse train of electrical signals may be part of an automated system (such as a robotic system). The applicator (pulse delivery device) or the pulse generator may be controlled by a software application, including through the user interface. The system may be a system (e.g., device or apparatus) for inactivating biofilms as described herein or may be any suitable system for applying sub-microsecond pulsed electric field to or into and/or in proximity to a surface to inactivate the biofilm including C. acnes cells.

In the treatment for acne, the sub-microsecond pulsed electric field may have an electric field strength like any described herein which is suitable for inactivating a bacterial biofilm. In some examples, the method may include use of an electric field strength from about 0.1 kV/cm to about 9.9 kV/cm; about 0.1 kV/cm to 5.0 kV/cm; about 0.5 kV/cm to 3.5 kV/cm; or any value therebetween. In some other examples, the method may include use of an electric field strength from about 0.1 kV/cm to about 75 kV/cm; about 0.1 kV/cm to 40 kV/cm; about 0.5 kV/cm to 30 kV/cm; about 1.0 kV/cm to 30 kV/cm; or any value therebetween. The pulse duration of the nanosecond pulses of the sub-microsecond pulsed electric field applied may be from about 1 nanosecond to about 900 nanoseconds; about 10 nanoseconds to about 500 nanoseconds; about 100 nanoseconds to about 400 nanoseconds; or any duration therebetween. In some examples, the pulse duration of the nanosecond pulse of the sub-microsecond pulsed electric field used to inactivate a biofilm of C. acnes may be a range of nanoseconds selected from any of the values described. In some examples, the method for treating acne may include a train of pulses of the sub-microsecond pulsed electric field from about 5 pulses to about 1000 pulses, or any number of pulses therebetween. In some examples, the sub-microsecond pulsed electric field may have a frequency from about 0.5 Hz to about 10 Hz, about 1 Hz to about 5 Hz, or any frequency therebetween. In some other examples, the sub-microsecond pulsed electric field may have a frequency from about 0.5 Hz to about 10 Hz; about 1 Hz to about 5 Hz; about 3 Hz to about 7 Hz; above 10 Hz, about 50 Hz, about 100 Hz, about 500 Hz, about 1 kHz, about 10 kHz, about 1 MHz, or for example, up to about 3 MHz, or any frequency therebetween. A method of treating acne may include any combination of the electric field strength, duration of pulses, number of pulses, configuration of the pulse train (e.g., how many pulses provided, time in between each pulse and momentary pauses to the pulse train), as can be determined by one of skill based on the teachings herein and that known generally in the art.

### Apparatuses for Inactivation of Bacterial Biofilms

Any apparatus (e.g., system, device, etc.) suitable for delivery of electrical pulses with the target energy level may be used. An apparatus (e.g., device or system) for inactivating biofilms may include a controller, a pulse generator, and a pulse delivery device (e.g., a wand, applicator, etc.). In some examples the controller may be part of or integrated with the pulse generator. The apparatus may include a power connector to electrically connect the apparatus to a source of power and a connector, such as a high voltage connector, to connect the pulse generator to the pulse delivery device. A pulse generator may be any pulse generator that is capable of generating pulses, for example, with a duration of 1,000 ns or less. Electrical energy may be delivered, for example, at high voltage power having a peak voltage of between 100 volts per centimeter (V/cm) and 100 kilovolts per centimeter (kV/cm) (e.g., greater than about 0.1 kV/cm greater than about 1 kV/cm, greater than about 5 kV/cm, greater than about 10 kV/cm, greater than about 20 kV/cm, greater than about 25 kV/cm, greater than about 40 kV/cm, greater than about 75 kV/cm, etc.). The pulse generator may generate sub-microsecond electrical pulses having a pulse width of between about 0.1 nanoseconds (ns) and less than 1000 nanoseconds (ns). In some examples, the nanosecond, e.g. sub-microsecond, pulsed electric field may have a frequency from about 0.5 Hz to about 10 Hz; about 1 Hz to about 5 Hz; about 3 Hz to about 7 Hz; above 10 Hz, about 50 Hz, about 100 Hz, about 500 Hz, about 1 kHz, about 10 kHz, about 1 MHz, or for example, up to about 3 MHz, or any frequency therebetween.

The pulse delivery device or an applicator may include a treatment tip for delivery of electrical therapy, and it may be partially or fully automated. The treatment tip may include one or a plurality of electrodes (e.g., two, four, or any other number), the electrodes may extend distally from the distal end of the treatment tip. The treatment tip may be removable (and in some configurations, single-use or low-use), and may be disposable or reusable, e.g., after cleaning and/or sterilizing. This pulse delivery device may include a control (button/switch) that sends a command signal to the controller (e.g., processor) to start and/or stop the apparatus. Multiple controls may be included. The one or more electrodes may be penetrating electrodes or non-penetrating surface electrodes, including without limitation, line electrodes, surface electrodes, needle electrodes, knife electrodes, flat electrodes, ring electrodes and the like. The electrodes may be coupled directly or indirectly, for example, to a motor or to a processor/controller. As stated above, the applicator/pulse delivery device may be a hand-held device or it may be operably connected to a movable arm (e.g., robotic arm) of an automated system.

The treatment tip may be configured for surface application of the nanosecond pulsed electrical fields. For example, the treatment tip may include an aperture (e.g., window, opening, etc.) which may be open on one side (or may be circumferential) in which the tissue to be treated may be positioned. The aperture may form a therapy region. Electrodes on the inner sides (e.g., faces) of the aperture may deliver a nanosecond pulsed electrical field to a biofilm on the tissue surface when the applicator is applied against the surface with the biofilm in the therapy region.

In some examples, the treatment tip is removable, and the electrodes may be held in place by a frame coupled to a treatment tip housing allowing the one or more electrodes to move relative to the tissue, and/or the tip housing, e.g., to rotate and/or translationally oscillate. In some examples, the electrodes may be connected to a linkage that is configured to connect to a driver.

The driver (e.g. motor, not shown) may be controlled by a manual or automatic control (e.g., button, switch, foot switch, finger button, toggle, etc.) on the pulse delivery device or apparatus to move the one or more electrodes. The control may in turn connect to a controller which may be internal to the pulse delivery device or separate, and may be the same controller controlling the application of electrical energy from the pulse generator, or it could be a different controller. In some examples a separate control for moving (e.g., rotating and/or mechanically oscillating) the electrodes and for applying energy may be provided. In some examples, the electrodes may be configured to prevent arcing by moving (e.g., oscillating and/or rotating, and/or non-oscillatory translation) while applying an energy therapy.

When multiple electrodes are included, they may be arranged as an array (e.g., a line, grid, parallel lines, etc.). In some examples, the driver is coupled to the one or more electrodes and is configured to rotate and/or translationally oscillate the one or more electrodes. When a plurality of electrodes is used, the electrodes may be jointly and/or individually moved.

Any of the apparatuses described herein may be used as part of a medical or a cosmetic procedure, including a minimally invasive surgical procedure. Any of the apparatuses described herein may be delivered or deployed through an endoscope, laparoscope, cannula, catheter, or the like. In particular, the apparatuses described herein may be used internally by application through an endoscope. Thus, any of these apparatuses may be operated with and/or may be integrated with an endoscope, catheter or similar tool. In operation, any of these devices may be inserted into a surgical incision and/or a port into a body region or body cavity.

Any of the methods (including user interfaces) described herein may be implemented as software, hardware or firmware, including as a non-transitory computer-readable storage medium storing a set of instructions capable of being executed by a processor (e.g., computer, tablet, smartphone, etc.), that when executed by the processor causes the processor to control performance or perform any of the steps, including but not limited to: displaying, communicating with the user, analyzing, modifying parameters (including timing, frequency, intensity, etc.), determining, alerting, or the like.

For example, any of these apparatuses may measure an electrical property, including tissue impedance, during or between pulsing. When the electrical property, such as impedance, changes by a predetermined amount, and/or at a predetermined rate of change, which may indicate that there is an increase susceptibility for arcing, the electrodes may be moved. The predetermined amount maybe determined empirically (e.g., by testing for arcing), and may be generic or may be tissue-specific. Once the electrical property changes relative to the predetermined amount the electrodes may be moved (e.g., rotated translated, etc.) or application of the energy may be stopped). The electrodes may be moved manually or automatically, including robotically. Change in impedance and/or the rate of change of impedance may be automatically detected by a processor or controller of the apparatus, and the same or a different processor or controller may trigger and direct the movement of the one or more electrodes.

Alternatively or additionally, a change in an electrical property of the tissue such as an impedance drop may also trigger a change in energy parameters, such as a change in pulse parameters. For example, in response to an impedance change (or rate of change) the apparatus may adjust the voltage applied (e.g., lowering the voltage), adjust the pulse width (e.g., decreasing the pulse width), and/or decreasing the pulse intensity (increasing the duration between pulses), and/or pausing pulsing until the tissue impedance recovers below the threshold.

Thus, the apparatuses or systems for inactivating a bacterial biofilm may further include a processor configured to monitor the electrical characteristic (e.g., impedance) of each pulse delivered, and to adjust the treatment based on the electrical characteristic to prevent or reduce arcing. For example, an apparatus, including a processor of the apparatus, and method of treating may be configured to monitor the rate of change of the impedance, or a related electrical characteristic, during or between each pulse or a train of pulses. For example, after delivery of some initial number of pulses, if the rate of change of the electrical characteristic increases above a threshold or drops below a threshold (e.g., an impedance rate change threshold) and/or the electrical characteristic changes by more than a threshold percent (e.g., impedance percent change threshold), and/or the electrical characteristic changes by a threshold magnitude (e.g., an impedance magnitude change threshold), the pulse settings may be adjusted to reduce, minimize or prevent arcing. The examples of detection of a change in an electrical characteristic of the tissue and potential adjustment described above are examples; more involved statistical algorithms could be used to assess the changes in impedance or other measured parameters. In any of these examples the number of pulses may be chosen manually or automatically, and or may be determined based on the treatment parameters.

The adjustment may be one or more of: changing the inter-pulse timing of the pulses (e.g., increase or decreasing the frequency of the pulsing, such as increasing the time between pulses), changing the pulse magnitude (e.g., current and/or voltage magnitude of the pulses), and/or changing the pulse duration (e.g., increasing and/or decreasing the pulse duration).

Any of the above-mentioned apparatuses, which may have any combination of features as described above, and the methods described herein may be implemented with a fully or partially automated system, for example, computer-controlled or robotic system. For example, a device for applying electrical therapy may be operatively attached or coupled to a robotic arm. The robotic system may include one or more electrodes, a robotic arm, and at least one processor/controller. The system may include a separate driver mechanism driving the movement (e.g., rotation, translational oscillation, etc.) of the one or more electrodes. However, in some examples, at least some of the movement of the applicator/pulse delivery device that comprises one or more electrodes may be directed by a robotic arm, for example, under control of the controller or processor of the robotic system. Therefore, the movements of the applicator and/or one or more electrodes may be directed by a driver mechanism of the applicator itself, a driver mechanism/controller of the robotic system, or combination of both. The at least one processor may control the movement of the robotic arm, the activation of the driver mechanism, or both. The same single processor may control all of the movements, or separate processors may direct the movement of the robotic arm and the driver mechanism. In some examples, the at least one processor (which may comprise one or a plurality of processors) may be operatively connected to a generator responsible for generating electrical pulses of the device for applying electrical therapy. The at least one processor may comprise instructions for implementing various methods described herein.

Various motors and other movement devices may be incorporated to enable fine movements of the device for delivery of electrical therapy and/or for operating the tip of the tool (device) so that it may be moved in multiple directions. A system including robotics may further include at least one (and preferably two for stereo vision, or more) image acquisition device which may be mounted in a fixed position, or coupled (directly or indirectly) to a robotic arm or other controllable motion device.

In those examples where image acquisition device is used, the processor may comprise an image processor for processing images obtained from the image acquisition device. The image processor may be a separate device or it may be incorporated as a part of the processor. The processor may also instruct the movements of the robotic arm, including the tool (device for delivery of electrical therapy), and act, for example, through a controller. The controller may be operatively coupled to the robotic arm and configured to control the motion of the robotic arm, including the motion based on the images or data acquired by the image acquisition device. Alternatively, controller may be incorporated as a part of a processor included within the apparatus, so that all processing and controls of all movements of all the tools, the robotic arm and any other moveable parts of the assembly, including those based on the images or data acquired by the image acquisition device, are concentrated in one place. The system may further comprise a monitor, mouse and keyboard. A magnified image of the tissue can be shown on the imaging display or monitor. In addition, the system may comprise other tools, devices and components useful in applying the electrical therapy. The system may further include an interface (not shown) adapted to receive an image data, various parts of the system allow an operator to monitor conditions and provide instructions, as needed. The processor may interact with the imaging device via the interface. The interface may include hardware ports, cables, leads, and other data transmission means, or it may comprise a computer program.

The processor may operate as a data processing device, for example, it may be incorporated into a computer. The processor may include a central processing unit or parallel processor, and input/output interface, a memory with a program, wherein all the components may be connected by a bus. Further, the computer may include an input device, a display, and may also include one or more secondary storage devices. The bus may be internal to the computer and may include an adapter for receiving a keyboard or input device or may include external connections.

The processor of apparatuses or systems for inactivating a bacterial biofilm may further or alternatively be configured to perform the methods describe herein. The processor may access the memory in which may be stored at least one sequence of code instructions comprising a program for performing the methods for inactivating a bacterial biofilm. The processor may execute a program that may be configured to include predetermined operations, or may execute a program configured to include customizable operations. The memory and the program may be located within the computer or may be located external thereto. By way of example, and not limitation, a suitable image processor may be a digital processing system which includes one or more processors or other type of device. For example, a processor and/or an image processor may be a controller or any type of personal computer (“PC”). Alternatively, the processor may comprise an Application Specific Integrated Circuit (ASIC) or Field Programmable Gate Array (FPGA). It will be understood by those of ordinary skill in the art that the processor and/or the image processor for use with the present disclosure is programmed and configured to perform various known image processing techniques, for example, segmentation, edge detection, object recognition and selection. Any of these techniques may be employed as is known in the art. The methods described herein may be implemented on various general or specific purpose computing systems. In certain examples, the methods of the present application may be implemented on a specifically configured personal computer or workstation. In other examples, the methods may be implemented on a general-purpose workstation, including one connected to a network. Alternatively or additionally, the methods of the disclosure may be, at least partially, implemented on a card for a network device or a general-purpose computing device. The processor and/or image processor may also include memory, storage devices, and other components as is known to one of skill in the art.

In some examples, the controller of the system may further include a machine-readable tangible medium storing instructions for causing the one or more processors to execute operations for: applying a train of sub-microsecond electrical pulses including an electric field strength, like any described herein, and may be from about 0.1 volts per centimeter (V/cm) and 100 kilovolts per centimeter (kV/cm), e.g., greater than about 1 kV/cm, greater than about 5 kV/cm, greater than about 10 kV/cm, greater than about 20 kV/cm, greater than about 25 kV/cm, greater than about 40 kV/cm, greater than about 75 kV/cm, or any value therebetween. In some other examples, the instructions may cause an electric field having a strength of about 50 kV/cm or 100 kV/cm. In yet other examples, the instructions may cause an electric field having a strength from 1 kV/cm to 9.9 kV/cm. In some examples, the instructions may include instructions for causing a pulse duration having a duration from about 1 nanosecond to about 900 nanoseconds; about 10 nanoseconds to about 500 nanoseconds; or about 100 nanoseconds to about 400 nanoseconds, any duration therebetween. In some examples, the instructions may include instructions for causing a pulse duration in a range of nanoseconds selected from any of the values described. In some examples, the instructions may include instructions causing a train of sub-microsecond electrical pulses from about 5 pulses to about 1000 pulses, or any number of pulses therebetween. In some examples, the instructions may include instructions causing a frequency from about 0.5 Hz to about 10 Hz; about 1 Hz to about 5 Hz; about 3 Hz to about 7 Hz; above 10 Hz, about 50 Hz, about 100 Hz, about 500 Hz, about 1 kHz, about 10 kHz, about 1 MHz, or for example, up to about 3 MHz, or any frequency therebetween.

The instructions may include any combination of the electric field strength, duration of pulses, number of pulses, configuration of the pulse train (e.g., how many pulses provided, time in between each pulse and momentary pauses to the pulse train), as can be determined by one of skill based on the teachings herein and that known generally in the art.

### EXAMPLES

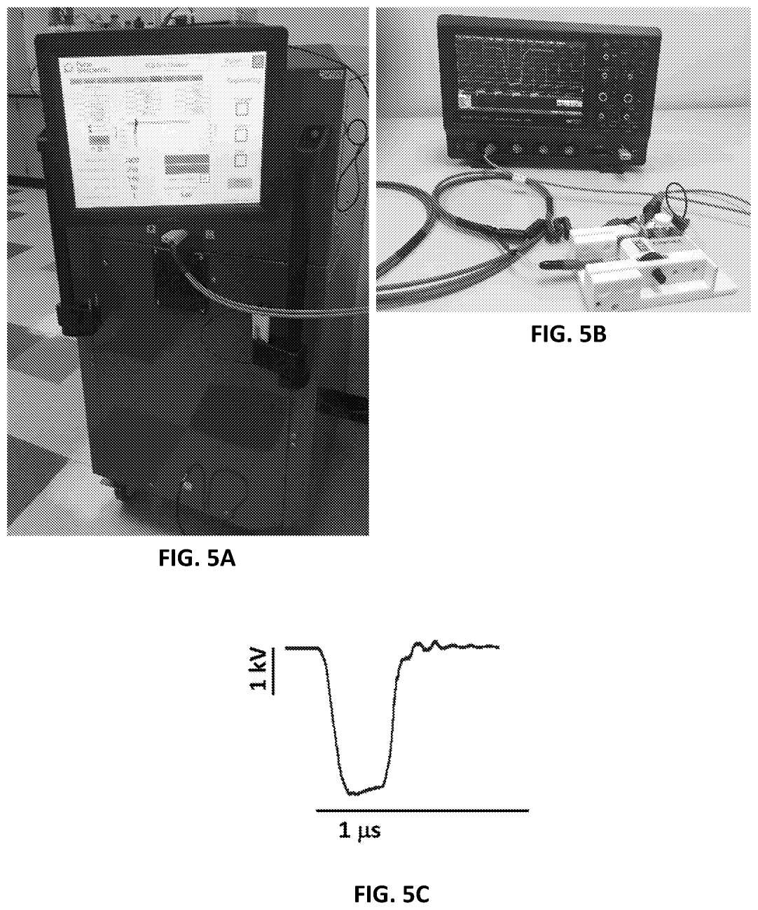

FIGS. 5A-5C illustrates one example of an apparatus for delivering nanosecond pulsed electrical fields as described herein. FIGS. 5A and 5B illustrate an example of a system configured to deliver a nanosecond pulsed electrical field. In FIG. 5A the controller and pulse generator are shown, including an interactive display that may be used to select the energy parameters applied. FIG. 5C illustrates one example of a pulse waveform, showing a trapezoidal pulse of 280 ns duration that may be produced by the pulse generation system and delivered to the applicator. In FIG. 5B the applicator shown is part of a 1 mm electroporation cuvette. In this example a digital oscilloscope was used to monitor the pulse amplitude and shape at the cuvette.

Any appropriate applicator may be used. For example, in FIGS. 5D and 5E, an example of a tissue-contacting applicator is shown. FIG. 5D shows a side view of the applicator 550 above a region of tissue (e.g., skin 563), including a handle region and a tissue-contacting region. The tissue-contacting region may be adjustably coupled to the handle region. In FIG. 5E, the therapeutic region is shown, formed between two arms or prongs, each having a tissue-contacting surface. In FIG. 5E, the first electrode 551 and the second electrode 553 are on inner surfaces of the window forming the skin-contacting surface. The nanosecond pulsed electrical field 555 may be applied between the electrodes, as shown.

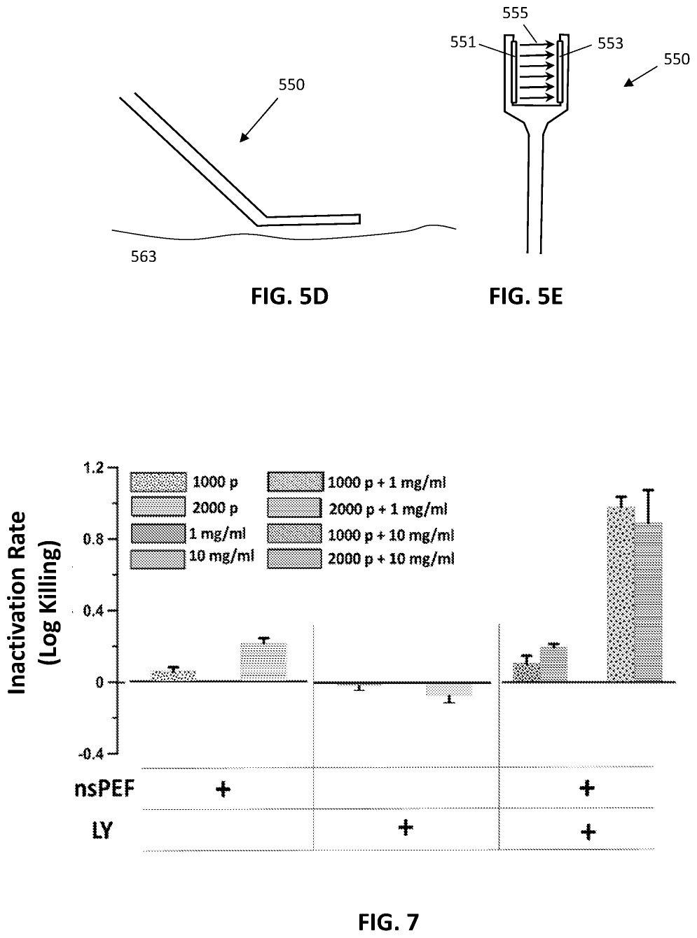

As illustrated in FIGS. 6A-6D, C. acnes (ATCC 29399) was grown in different medium. The cells were directly streaked into the respective media and incubated at 37° C. in an anaerobic chamber with an atmosphere of 85% N₂, 10% CO₂ and 5% H₂ (Coy Laboratory Products, Grass Lake, Mich.). The growth of the cells was monitored at 72 hr intervals after the start of incubation. Tiny, round, mucoid and opaque colonies of C. acnes were observed at 72 hrs. after plating. Among these TY medium containing 3% peptone, 2% yeast extract, 0.1% sodium thioglycolate pH balanced at 7.4 showed high consistent colony counts and therefore was chosen for culturing C. acnes.

Furthermore, in order to avoid possible contamination from other anaerobic organisms and increase the selectivity, the TY media was supplemented with 20 ug/mL of metronidazole (BTC CAS:443-48-1) since C. acnes is inherently resistant to the antibiotic. A growth curve assay was done by measuring optical density at 600 nm (OD₆₀₀) at multiple intervals for 72 h. All plastic supplies were allowed to equilibrate for at least 72 hrs. in the anaerobic chamber.

Experiment 1. Electric pulse treatment and viability assessment of C. acnes in suspension and in scraped biofilms. C. acnes starter culture was prepared by growing the cells in TY+Met20 media for 48 hrs at 37° C. in an anaerobic chamber. Starter cultures were diluted 1:10 into fresh growth medium and cultured for 4-6 hours, which was experimentally determined to allow entry into exponential growth phase (log phase). Alternately, cells were grown in sterile plastic 24 well plates coated with poly-L-lysine for 72 hours. Non-adherent cells were removed by pipetting and washing with sterile growth medium, and then adherent biofilm-derived cells were scraped from the bottom, resuspended in fresh growth medium, and vortexed to homogeneity. All samples were aliquoted into airtight 1 mm gap electroporation cuvettes (BioSmith, San Diego, Calif.). Cuvettes containing the cells were wrapped with parafilm and removed from the anaerobic chamber. Cuvettes with planktonic log phase cells or biofilm derived cells were treated at room temperature (22±2° C.) with respective up to 2000 electric pulses (including 200 pulses, 500 pulses, 1000 pulses, etc.) using PulseTX pulse generator (Pulse Biosciences, Inc). In this experiment, samples were exposed to the 28 kV/cm of trains of trapezoidal 280-ns pulses at 5 Hz. Technical duplicates were prepared for each of the treatments including the control, with triplicate experiment repetition. Control samples, in which cells were aliquoted into cuvettes and transported with the experimental samples but not treated were prepared for all experiments. After the treatment, the cuvettes were returned into the anaerobic chamber where the control and experimental samples were serially diluted to 10⁻⁵ dilution in the growth medium and 100 uL of each of the samples were spread on selective TY-metronidazole plates. The plates were incubated in the incubator at 37° C. in the anaerobic chamber for 72 hrs, after which the visible colony forming units (CFU) were counted for the treated and untreated samples to determine the loss of CFU due to treatment.

Growth of Biofilm and aged planktonic C. acnes: C. acnes cells were grown in TY media and allowed to form biofilm by growing them for 72 hrs on poly-L-lysine coated plastic growth plates as described for 72 hours. Planktonic cells from the non-adherent fraction were pipetted out from the top of the biofilm without disturbing the biofilm. The remaining biofilm-coated plastic growth plates were rinsed with fresh media to remove remaining traces of planktonic C. acnes cells, and then the C. acnes-containing biofilm was scraped from the plastic growth plates and aliquoted to cuvettes in fresh medium.

Cuvettes were treated with respective electric pulses including 250 pulses, 500 pulses, 1000 pulses and 2000 pulses using PulseTX pulse generator (Pulse Biosciences, Inc), using trains of trapezoidal 300-ns pulses at 28 kV/cm, 5 Hz. Untreated controls were included for all experiments. Technical duplicates were prepared for each of the treatments including the control, with triplicate experiment repetition. Immediately after treatment, serial dilutions of the suspension ranging from 10⁻¹ to 10⁻⁷ were made. At 72 hrs post treatment, treated and untreated CFU were counted and compared.

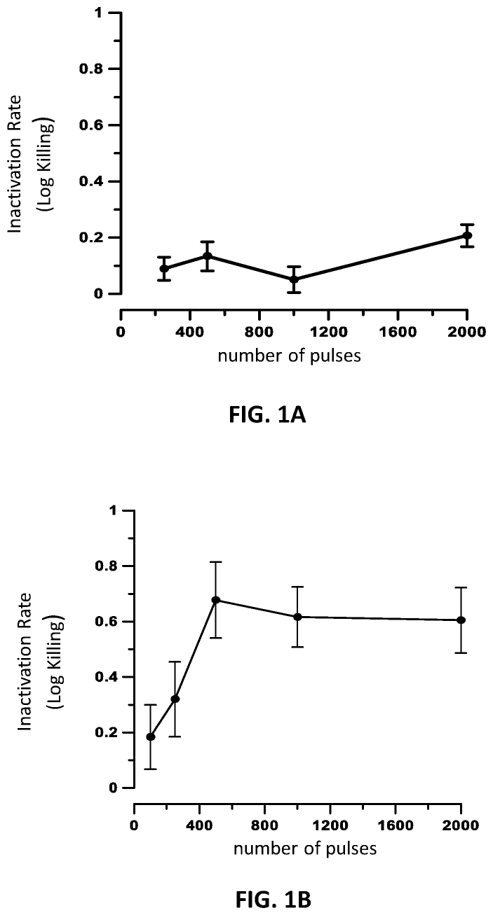

In FIG. 1A, the results for sub-microsecond (e.g., nanosecond) pulsed electric field efficacy at killing planktonic cells (e.g., C. acnes cells grown in suspension) are shown. Even at the highest treatment level, 2000 pulses at 28 kV/cm, 5 Hz, the extent of cell killing is low, reaching only 0.2 log killing. In contrast, the scraped biofilm-contained C. acnes cells, subjected to the same increasing series of trains of pulses, are far more susceptible to killing by this mechanism. The results show that while all tested sub-microsecond pulsed electric field doses failed to inactivate planktonic C. acnes (FIG. 1A), they significantly impaired bacteria in biofilms (FIG. 1B). Maximum cell killing was achieved with over about 500 of pulses and a 0.6 log killing. Data in FIGS. 1A-1B are expressed at a log 10 number differences between untreated control and the sub-microsecond pulsed electric field-treated samples. Mean+/−s.e., n=5-8.

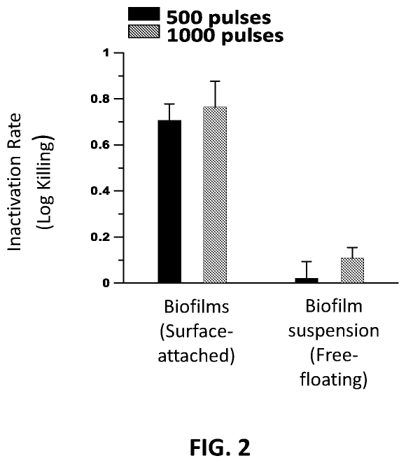

Experiment 2. Electric pulse treatment and viability of planktonic cells compared to that of biofilm-contained cells. To determine whether planktonic cells and biofilm-contained cells grown under the same conditions still exhibited differential sensitivity to sub-microsecond pulsed electric field (e.g., nanosecond), biofilms were allowed to grow in TY medium for 72 hours in poly-L-lysine coated 24 well plates as described; the planktonic C. acnes cells from the non-adherent fraction were removed by pipetting. Adherent biofilms were washed, scraped out of the biofilms, and vortexed to homogeneity as previously described. The age-matched planktonic and biofilm-derived cells were aliquoted into cuvettes for treatment as previously described. Each set of cuvettes were treated with sub-microsecond pulsed electric field, with 500 or 1000 pulses, using trains of trapezoidal 300-ns pulses at 28 kV/cm, 5 Hz. Untreated controls were included for all experiments. After the treatment, the cuvettes were returned into the anaerobic chamber where the cells were serially diluted between 10⁻¹ to 10⁻⁷ dilution in the growth medium and 100 uL of each of the samples were plated into selective TY media plates. The plates were incubated in the incubator at 37° C. in the anaerobic chamber for 72 hrs, after which the visible colony forming units (CFU) were counted for the treated and untreated samples. Mean+/−s.e., n=3-8. As shown in FIG. 2, the C. acnes cells of the biofilm were significantly more susceptible to cell killing and demonstrated greater than about 0.7 log killing for both 500 pulses and 1000 pulses, while the C. acnes cells in suspension (e.g., planktonic cells) were little affected, with a log killing of 0.1 or less. While the phenotype of planktonic cells (e.g., in suspension in the presence of biofilm) in this experiment are thought to be at least partially differentiated from planktonic cells grown completely in suspension, e.g., not in the presence of a biofilm, the planktonic cells of this experiment exhibited striking differences in behavior from that of the biofilm-forming C. acnes cells. The planktonic cells appear to be much less susceptible to sub-microsecond pulsed electric field, as shown in FIG. 2.

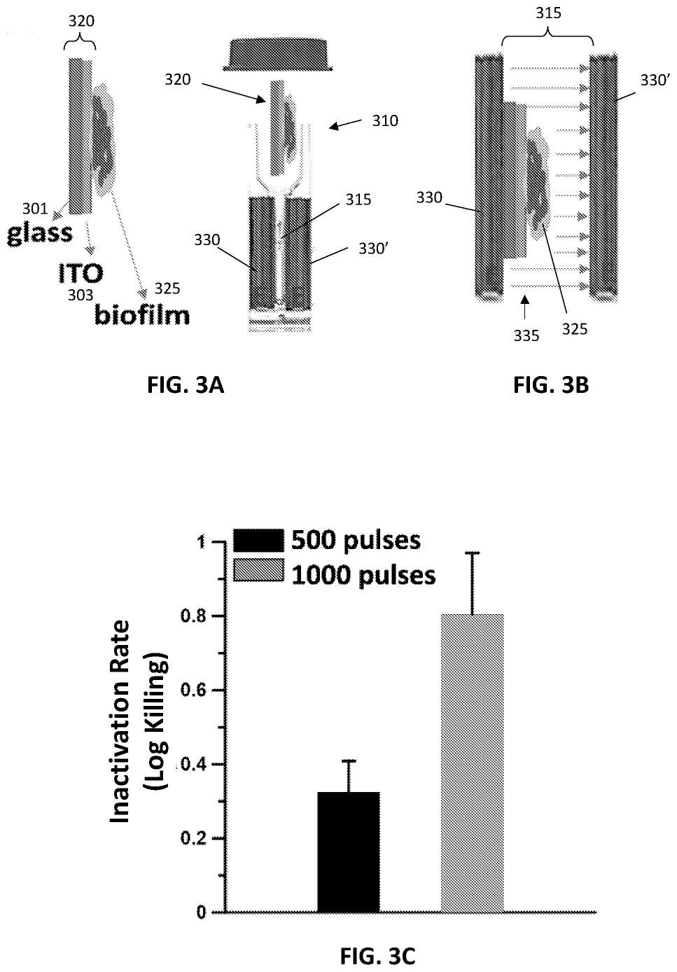

Experiment 3. Effect of sub-microsecond pulsed electric field on undisrupted biofilms. To remove the possibility that scraping the C. acnes cells contained within the biofilm altered their susceptibility to sub-microsecond pulsed electric field treatment, C. acnes cells grown within a biofilm were tested without abrasive manipulation. C. acnes cells were grown on glass coverslips 301 having an indium oxide (ITO) conductive layer 303 for 72 hrs. as above, using the same media. Planktonic cells were rinsed away from the glass coverslip. The coverslip 320 was aseptically placed into a standard electroporation cuvette 310, without scraping or disrupting the biofilm. Stressful cell handling and possible confounding impact of detachment of the cells was therefore eliminated. FIG. 3A shows the insertion of the ITO coverslip 320 containing the undisturbed, rinsed biofilm of C. acnes cells as it was moved into the region 315 between electrodes 330, 330′ of the electroporation cuvette. FIG. 3B shows the disposition of the ITO coverslip 320 within region 315. The biofilm 325 was subjected to the pulsed electric field as shown by the arrows 335 extending from the first electrode 330 (bottom of figure) to the second electrode 330′ (top of figure). Each set of cuvettes were treated with sub-microsecond pulsed electric field, with 500 or 1000 pulses, using trains of trapezoidal 280-ns pulses at 3.6 kV/cm, 5 Hz. After treatment, the cuvettes were returned into the anaerobic chamber where the cells were scraped from the coverslip 320 into fresh growth medium and vortexed to homogeneity and then serially diluted to 10⁻⁵ dilution in the growth medium and 100 uL of each of the samples were plated into selective TY-metronidazole media plates. The plates were incubated in the incubator at 37° C. in the anaerobic chamber for 72 hrs, after which the visible colony forming units (CFU) were counted. The data is shown in FIG. 3C, where better than 0.3 log killing was observed with the 500 pulse train, and about 0.8 log killing was observed with the 1000 pulse train. The extent of killing is remarkable at the 3.6 kV/cm electric field strength, which is about 13% of the electric field strength used in Experiments 1 and 2. Data are expressed as log 10 number differences between untreated control and sub-microsecond pulsed electric field-treated samples. Mean+/−s.e., n=9. This demonstrates that very low electric field strength pulses are sufficiently strong to damage and kill the bacteria within the biofilms, relative to the strength of electric field needed to kill or significantly damage planktonic bacteria not within the biofilm.

Experiment 4. Effects of pretreatment with lysozyme. As Experiments 1-3, showed a surprising differentiated response to sub-microsecond pulsed electric field between planktonic cells and biofilm-contained bacterial cells, pretreatment with a cell wall weakening agent, lysozyme, was studied. Lysozyme pre-treatment was examined to determine whether increased susceptibility to NPS for planktonic bacterial cells could be obtained. This is particularly desirable in applications, such as acne treatment, where commensally living planktonic C. acnes are not beneficial, and inactivation of both biofilm-contained and planktonic C. acnes cells is sought. In order to assess the combined effect of lysozyme and sub-microsecond pulsed electric field on C. acnes viability, planktonic cells and biofilms were pretreated with either 1 mg/ml lysozyme or 10 mg/ml lysozyme (MP Biomedicals CAS 9001-63-2) for 1 hour at 37° C. in an anaerobic chamber prior to electric field application.

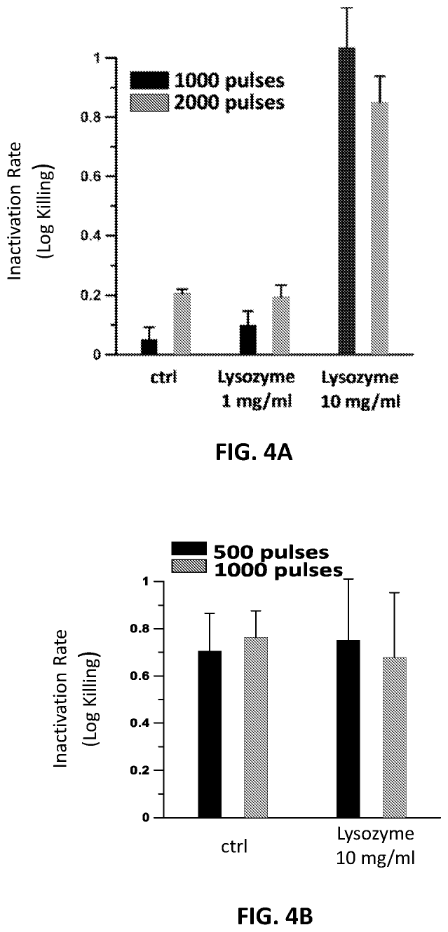

Planktonic C. acnes cells in in exponential phase were treated with either 1 or 10 mg/ml lysozyme. After 1-hour, cells were treated with sub-microsecond pulsed electric field (1000-2000, 280 ns pulses, 28 kV/cm, 5 Hz). Viability was measured at 72 h post treatment, which was performed as described above in Experiment 1. The results are shown in FIG. 4A. Pre-treatment of planktonic cells at a concentration of 1 mg/ml did not produce significant improvement in susceptibility, compared to controls (sub-microsecond pulsed electric field only). However, at the higher concentration of 10 mg/ml, susceptibility to sub-microsecond pulsed electric field was effectively enhanced. Whether a 1000 pulse or 2000 pulse train was used, planktonic C. acnes cell populations were significantly reduced, showing a log 10 killing of about 1.0 for sub-microsecond pulsed electric field using 1000 pulses and over about 0.8 log 10 killing for sub-microsecond pulsed electric field using 2000 pulses.

In contrast, biofilm-contained C. acnes cells, even when pretreated with the higher 10 mg/ml concentration of lysozyme, did not exhibit increased susceptibility, compared to controls treated only with ns-PEF, as shown in FIG. 4B. The pulse trains used for biofilm contained C. acnes cells were 500 or 1000, 280 ns pulses, 28 kV/cm, 5 Hz. Viability was determined at 72 h post sub-microsecond pulsed electric field and performed as described above in Experiment 1. Without being bound by any theory, the thick extracellular matrix of biofilm-contained bacterial cells, which is known to be resistant to drug penetration, may require an even higher concentration of lysozyme to produce a significant effect in enhancing susceptibility.

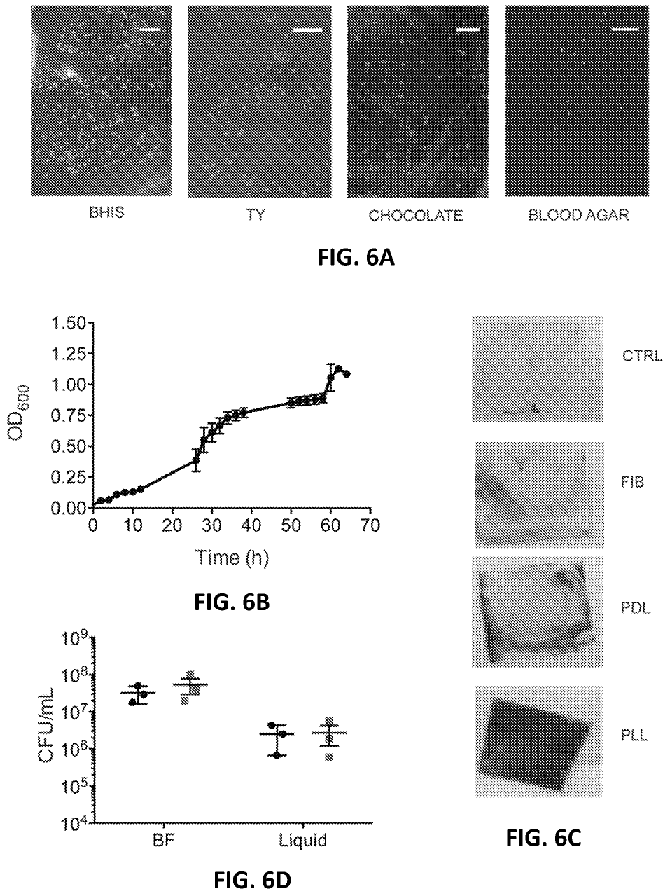

Incubation of C. acnes supernatant with lysozyme (LY) at various concentrations reduces C. acnes activity, and LY-triclosan complexes significantly enhance bactericidal activity against several strains of Gram-positive and Gram-negative bacteria. Destabilizing the cells wall of C. acnes with LY increase cells sensitivity to sub-microsecond pulsed electric fields, as illustrated in FIG. 7. In this example, planktonic C. acnes cells were treated with 0, 1 or 10 mg/ml LY at 37 degrees C. After 1 h, samples were either exposed to 280 ns pulses (1000 or 2000, 28 kV/cm, 5 Hz) or left untreated as parallel sham controls, and colonies were counted at 72 h post treatment. These results show that treatment with either sub-microsecond pulsed electric fields alone or lysozyme alone did not affect C. acnes viability. Indeed, lysozyme increased C. acnes growth suggesting that perturbation of the cell wall may accelerate cell division. However, combining a pretreatment of C. acnes with 10 mg/ml LY with sub-microsecond pulsed electric fields significantly increased C. acnes inactivation. These results reveal for the first time a synergistic effect between lysozyme and sub-microsecond pulsed electric fields at killing Gram-positive bacteria. Note that although many of the examples described herein were performed at about 280 ns, other pulse widths may be used successfully, including, for example about 10 ns, about 50 ns, about 100 ns, about 300 ns, about 400 ns, about 500 ns, etc. (e.g., between 1 ns and about 1000 ns, e.g., between about 1 ns and about 900 ns, between about 1 ns and about 800 ns, between about 1 ns and about 600 ns, between about 1 ns and about 500 ns, between about 10 ns and about 500 ns, between about 50 ns and about 500 ns, between about 100 ns and about 500 ns, etc.).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or examples of various examples. Combinations of the above examples, and other examples not specifically described herein, will be apparent to those of skill in the art upon reviewing the above description.

1-30. (canceled)

31. A system for inactivating a bacterial biofilm, the system comprising:
an applicator comprising a set of electrodes configured to be placed in proximity to the bacterial biofilm;
a pulse generator configured to generate a sub-microsecond pulse; and
a controller comprising one or more processors, wherein the controller comprises a machine-readable medium storing instructions that, when executed by the one or more processors, cause the pulse generator to apply a train of sub-microsecond electrical pulses to the applicator to disrupt the bacterial biofilm on a tissue surface without damaging co-habiting commensal bacteria as well as limiting damage to the tissue when the bacterial biofilm is between the set of electrodes,
wherein the train of sub-microsecond electrical pulses has an electric field strength of between 0.1 kV/cm and 9.9 kV/cm.

32. The system of claim 31, wherein the applicator comprises a first contacting surface adjacent to a first electrode, a second contacting surface adjacent to a second electrode, and a therapy region between the first and the second electrodes, and wherein the applicator is configured to apply the electric field between the first and the second electrodes.

33. The system of claim 31, wherein the instructions cause the applicator to apply the electric field having a strength from 0.1 kV/cm to 5.0 kV/cm.

34. The system of claim 31, wherein the instructions cause the applicator to apply the train of sub-microsecond electrical pulses having a pulse duration from 10 nanoseconds to 900 nanoseconds.

35. The system of claim 31, wherein the instructions cause the applicator to apply the train of sub-microsecond electrical pulses having a pulse duration from 10 nanoseconds to 500 nanoseconds.

36. The system of claim 31, wherein the instructions cause the applicator to apply the train of sub-microsecond electrical pulses having from 10 pulses to 1000 pulses.

37. The system of claim 31, wherein the instructions cause the applicator to apply the train of sub-microsecond electrical pulses having a frequency from 0.5 Hz to 3 MHz.

38. The system of claim 31, wherein the instructions cause the applicator to apply the train of sub-microsecond electrical pulses having a frequency from 1 Hz to 10 Hz.

39. A method of inactivating a bacterial biofilm on a tissue, the method comprising applying a sub-microsecond pulsed electric field comprising an electric field strength from 0.1 kV/cm to 9.9 kV/cm to a surface of the tissue comprising the bacterial biofilm until the bacterial biofilm is inactivated, while limiting inactivation of planktonic bacteria in proximity to the bacterial biofilm and damage to the tissue.

40. The method of claim 39, wherein the sub-microsecond pulsed electric field comprises from 5 pulses to 1000 pulses.

41. The method of claim 39, wherein the sub-microsecond pulsed electric field comprises an electric field strength from 0.1 kV/cm to 5.0 kV/cm.

42. The method of claim 39, wherein the sub-microsecond pulsed electric field comprises an electric field strength from about 0.5 kV/cm to about 3.5 kV/cm.

43. The method of claim 39, wherein the sub-microsecond pulsed electric field has a pulse duration from 10 nanoseconds to 500 nanoseconds.

44. The method of claim 39, wherein the sub-microsecond pulsed electric field has a frequency from 0.5 Hz to 3 MHz.

45. The method of claim 39, further comprising contacting the surface comprising the bacterial biofilm with lysozyme prior to applying the sub-microsecond pulsed electric field.

46. The method of claim 45, wherein contacting the surface comprising the bacterial biofilm comprises contacting the surface with lysozyme at a concentration from at least 1 mg/ml to 10 mg/ml in a liquid medium.

47. The method of claim 45, wherein the surface comprising the bacterial biofilm is contacted with lysozyme for a period of time from 10 min to 1 h.

48. The method of claim 39, wherein the method is for treating acne.

49. A method of treating acne in a region of skin of a subject, comprising applying a sub-microsecond pulsed electric field to the region of skin comprising a C. acnes bacterial biofilm, wherein the sub-microsecond pulsed electric field is applied at an electric field strength, sufficient to inactivate the C. acnes bacterial biofilm while limiting inactivation of co-habiting commensal bacteria and damage to surrounding tissues in the region of skin.

50. The method of claim 49, wherein the sub-microsecond pulsed electric field comprises an electric field strength from 0.1 kV/cm to 9.9 kV/cm.

51. The method of claim 49, wherein the sub-microsecond pulsed electric field has a frequency from 0.5 Hz to 10 Hz.
